(19) United States
(12) Patent Application Publication
(10) Pub. No.: US 2017/0274026 A1
(43) Pub. Date: Sep. 28, 2017

(54) METHOD OF MAKING TRITERPENOIDS FROM PETRI DISH CULTUREDANTRODIA CINNAMOMEA

(71) Applicant: Ultra-Microrigin Biomedical Technology Co., Ltd., Zhubei City (TW)

(72) Inventors: Ming-Hsi Chuang, Zhubei (TW); Chiu-Ying Peng, Zhubei (TW); Hsin-Yu Chung, Zhubei (TW); Cheng-Yu Chi, Zhubei (TW); Chu-Ting Liu, Zhubei (TW); Hui-Chen Kuo, Zhubei (TW)

(21) Appl. No.: 15/134,482

(22) Filed: Apr. 21, 2016

(30) Foreign Application Priority Data
Mar. 25, 2016 (TW) 1051019485

(51) Int. Cl. A61K 36/07; G01N 33/50

(52) U.S. Cl. CPC A61K 36/07; G01N 33/5011; A61K 2236/37; A61K 2236/55

(57) ABSTRACT

A method of making triterpenoids from petri dish cultured Antrodia cinnamomea includes: A. providing petri dish cultured Antrodia cinnamomea in an extracting container; B. providing a supercritical solvent to the extracting container to obtain an Antrodia cinnamomea extract; sending the Antrodia cinnamomea extract to a chromatographic column to separate impurities and triterpenoids from the Antrodia cinnamomea extract, and removing the impurities, and collecting the triterpenoids containing fraction; and C. testing the bioactivity of the triterpenoids containing fraction by cytotoxicity test, cell morphology analysis, cell cycle and apoptosis test, and cell motility test to find an anti-cancer effect of the triterpenoids.

### BACKGROUND OF THE INVENTION

1. Technical Field

The present invention relates generally to active components of petri dish cultured Antrodia cinnamomea, and more particularly to a method of making triterpenoids from petri dish cultured Antrodia cinnamomea, which is effective in anti-cancer.

2. Description of Related Art

Fruiting bodies of Antrodia cinnamomeain dude various components, and some of the active components thereof, such as MMH01 (dehydrosulphurenic acid) and MMH02 (lanostane triterpenoids), are effective in anti-tumor and repressing the growth of cancer cells, see Toxicology in Vitro and International Journal of Gerontology. Petri dish culturing is a conventional way of growing Antrodia cinnamomea. It has many advantages, including fast growing, good anti-cancer effect, and reducing the chopping of Cinnamomum kanehirae.

There are patents disclosing the anti-cancer components of Antrodia cinnamomea, and methods making the anti-cancer components, such as CN102232944B, CN102000047B, CN102232945B, CN102232940B, CN102232943B, CN102232942B, CN102232941B, CN102443613B, CN104177240A, and U.S. Pat. No. 8,546,366. Those patents disclosed that the components in the extract of Antrodia cinnamomea are effective in anti-cancer.

There are patents disclosing the methods of extracting triterpenoids from Antrodia cinnamomea as well. CN102614195A and US2010/0210869A1 disclose a method of extracting ergosterol and lanosterol from fruiting bodies of Antrodia cinnamomea by ethanol and hexane (or ethyl acetate). U.S. Pat. No. 7,994,158 discloses a method, including extracting by water or an organic solvent, such as acetic acid, ethyl acetate, benzene, alcohol, and methylene dichloride, and then absorbing, concentrating, and purifying the extract by silicone and Sephadex. CN101555436B and TWI487531 disclose a method of extracting by ethanol and supercritical carbon dioxide. With the last method, ethanol and supercritical carbon dioxide are sent to a container, in which solid Antrodia cinnamomea is received. However, it is hard to reach a steady condition, or the active components are extracted unstably. Even it is performed by solid-liquid separation and bath extracting, it can't get purified extract, and it has a high cost.

In conclusion, most of the conventional methods of extracting active components from Antrodia cinnamomea use organic solvents to extract, and then perform the steps of concentration, separation, and purification in sequence. They usually have several drawbacks, such as complex processes, residual organic solvent, and toxicity problem. The conventional method of extracting by supercritical liquid directly uses the mixed solvent and carbon dioxide that need a lot of tests and work experiences, and furthermore, it needs the step of purification.

### BRIEF SUMMARY OF THE INVENTION

In view of the above, the primary objective of the present invention is to provide a method of making triterpenoids from petri dish cultured Antrodia cinnamomea, which may reach the stable condition and maximum separation with less ethanol and supercritical carbon dioxide in a supercritical status. With various cell tests, it may prove that triterpenoids is effective in anti-cancer. The present invention is a safe, recycling, and power saving method.

The present invention provides a method of making triterpenoids from petri dish cultured Antrodia cinnamomea, including A. providing 1-2 Kg petri dish cultured Antrodia cinnamomea in an extracting container; B. providing a supercritical solvent to the extracting container to obtain an Antrodia cinnamomea extract; after 30-60 minutes, sending the Antrodia cinnamomea extract to a chromatographic column to separate impurities and triterpenoids from the Antrodia cinnamomea extract, and then removing the impurities at a bottom of the chromatographic column, and collecting the triterpenoids containing fraction at a top of the chromatographic column, wherein the supercritical solvent is a saturated mixture of supercritical carbon dioxide and ethanol with a volume ratio of 1:0.1-0.2 (v/v); a pressure of the supercritical solvent is set to 2,000-4,000 psi, a temperature is set to 40-60° C., and a flow rate is set to 3-9 L/hr; and C. testing the triterpenoids containing fraction by cytotoxicity test, cell morphology analysis, cell cycle and apoptosis test, and cell motility test to find an anti-cancer effect of the triterpenoids containing fraction.

The present invention further provides triterpenoids containing fraction, which is effective in anti-cancer, made by the method as described above.

### BRIEF DESCRIPTION OF THE SEVERAL VIEWS OF THE DRAWINGS

The present invention will be best understood by referring to the following detailed description of some illustrative embodiments in conjunction with the accompanying drawings, in which

FIG. 1 is a flow chart of a preferred embodiment of the present invention;

FIG. 2 is a HPLC diagram, showing triterpenoids making by the method of the present invention and triterpenoids making by ethanol;

FIG. 3 is a diagram, showing (A) the viability of HCT116 cells treated with ethanol extracted Antrodia cinnamomea at different concentrations, and (B) the viability of HCT116 cells treated with the triterpenoids containing fraction at different concentrations after 48 hours;

FIG. 4 is pictures showing morphological change in control HCT116 cells, ethanol extracted Antrodia cinnamomea treated group, and the triterpenoid containing fraction treated group after a predetermined time;

FIG. 5 is cell-cycle diagrams showing the cell distribution in cell cycle in control HCT116 cells, ethanol extracted Antrodia cinnamomeat treated group, and the triterpenoid containing fraction treated group after a predetermined time; and

FIG. 6 is pictures showing cell motility of control HCT116 cells, ethanol extracted Antrodia cinnamomea treated group, and the triterpenoid containing fraction treated group after 0 and 24 hours.

### DETAILED DESCRIPTION OF THE INVENTION

The detailed description and technical contents of the present invention will be explained with reference to the accompanying drawings. However, the drawings are for illustration only and cannot be used to limit the present invention.

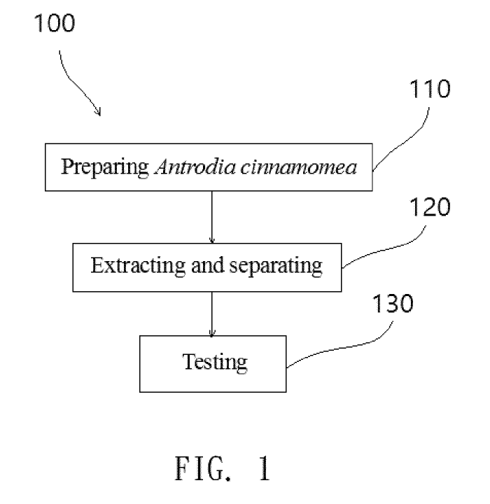

As shown in FIG. 1, a method 100 of making triterpenoids, which is effective in anti-cancer, from petri dish cultured Antrodia cinnamomea by the preferred embodiment of the present invention includes the following steps:

The first step 100 is preparing Antrodia cinnamomea 110, including putting 1-2 Kg petri dish cultured Antrodia cinnamomea in an extracting container. The extracting container is a stainless column with a diameter in a range between 0.036 m and 0.125 m and a height of 0.5 m.

The second step is extracting and separating 120. This step is performed under 2,000-4,000 psi and 40-60° C. A supercritical solvent with a flow rate of 3-9 L/hr is applied to the extracting container for extracting. The supercritical solvent is a saturated mixture of supercritical carbon dioxide and ethanol with a volume ratio of 1:0.1-0.2 (v/v) to obtain an Antrodia cinnamomea extract. After 30-60 minutes, apply the Antrodia cinnamomea extract to a chromatographic column to separate impurities and triterpenoids containing fraction from the Antrodia cinnamomea extract, and then remove the impurities at the bottom of the chromatographic column, and collect the triterpenoid-rich fraction at the top of the chromatographic column.

The chromatographic column is made of stainless, having an interior diameter of 0.036-0.125 m and a height of 1 m. A stainless plate is received in the chromatographic column, which is made of Pro-Pak protruded metal, saddles, rings, structured packing, or knitted packing.

The third step is testing 130, in which the triterpenoids containing fraction is tested by cytotoxicity test, cell morphology analysis, cell cycle and apoptosis test, and cell motility test to evaluate the anti-cancer effect of the triterpenoid containing fraction.

In the cytotoxicity test, HCT116 cells are put into a 96-well culture plate (a culture plate having 96 wells, and each well receives 10,000 HCT116 cells), in which a 200 μL complete medium (McCoy's 5a) is received. The complete medium is replaced by a triterpenoids medium (a medium having the triterpenoid containing fraction at a concentration of 0.25-1.0 mg/mL) in the triterpenoid containing fraction treated group next day. The control group is replaced with 200 μL complete medium only. After 2 days, a ratio of a viable cell count in each well is tested by a (3-(4,5-dimethylthiazol-2-yl)-2,5-diphenyltetrazolium bromide (MTT) colorimetric assay at an optical density of 570 nm by an ELISA reader.

In cell morphology analysis, HCT116 cells are received in a petri dish, and replaced with the triterpenoids medium (1.0 mg/mL) next day. The petri dish is kept and pictured by an inverted microscope at different times (2, 7, 14, 21, and 28 days).

In cell cycle and apoptosis test, HCT116 cells are treated with the triterpenoids medium (1.0 mg/mL) for a predetermined time, harvested with trypsin-EDTA, and then neutralized with the complete medium to obtain a solution. Next the solution is centrifuged to remove supernatant liquor thereof, washed by phosphate buffered saline, added to 1 mL 70% cool methanol, kept at 4° C. environment for a night, centrifuged again, then washed with 1 mL phosphate buffered saline for suspension, and then added with 50 mg/mL propidiumiodide for a 10 minutes photophobic process. The cell distribution in cell cycle is collected by an excitation beam at 532 nm and an emission beam at 590±40 nm by a flow cytometry.

In cell motility test, HCT116 cells are put in a 6-well culture plate (a culture plate having 6 wells, and 8,000 HCT116 cells are received in each well), and added with a 2 mL complete medium (McCoy's 5a). Next day, a line is drawn on confluent cells by a 200 μL pipette tip, and culture medium is replaced with the triterpenoids medium (0.25-1.0 mg/mL), and pictures of the wound drawn are taken on both Day 0 and Day 1 under a 100× magnification microscope. The control group has a 2 mL complete medium only without any addition, and is kept for 24 hours to take pictures for the measurement of the change in wound width between Day 0 and Day 1. At last, a measurement software (Image J) is applied to measure wound width between 5 opposite points (random) on each picture, and at least 25 data is collected for each conditions to compare averages motility between conditions.

The following description is about the tests of the triterpenoids obtained by the method of the preferred embodiment of the present invention, and the test of the effect of triterpenoids obtained by the preferred embodiment of the present invention on anti-cancer.

First, the measurement triterpenoid content is carried out on the conventional ethanol extract and the triterpenoids containing fraction of the present invention. Ursolic acid is applied as a standard sample, a content of the standard sample is obtained by measuring an optical density (mg/mL) of 548 nm. A measurement of components and contents of the triterpenoids is carried out by a quantity analysis with C18 column of HPLC, and the unit is mg/mL.

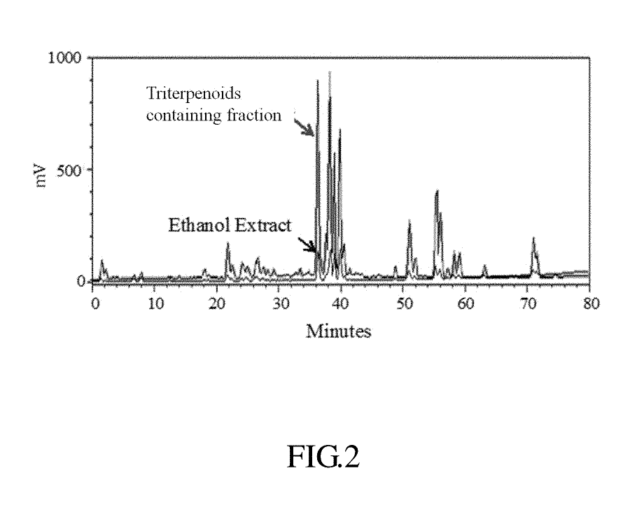

As shown in FIG. 2, the triterpenoid content can be compared by the area under peak curvatures of the ethanol extract and the triterpenoid containing fraction. The result shows that the peak of the triterpenoid containing fraction is higher than that of the ethanol extract, which means that the triterpenoid content of the triterpenoid containing fraction is much higher than that of the ethanol extract.

The tests of effect of the triterpenoid containing fraction on the anti-cancer are described hereafter:

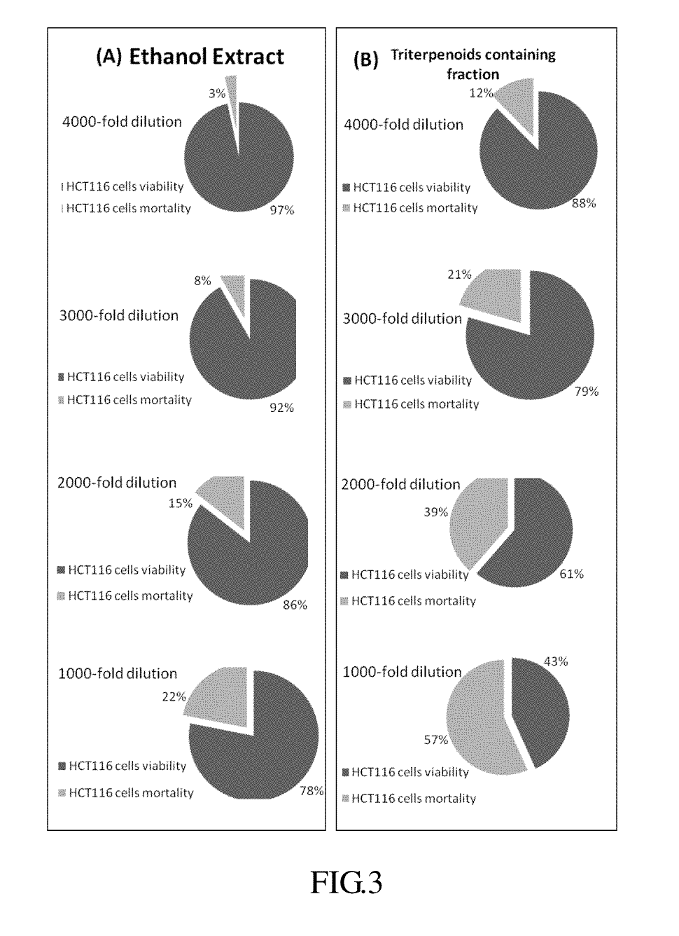

FIG. 3(A) shows the viability of HCT116 cells treated with the ethanol extracts at different concentrations, and FIG. 3(B) shows the viability of HCT116 cells treated with the triterpenoid containing fraction of the present invention at different concentrations. The results shows that the viability of HCT116 cells in the triterpenoid containing fraction of the present invention is lower than that in the ethanol extract treated group at the same concentration, which means that the present invention is more effective in killing cancer cells than the prior art. In comparison with the viability of HCT116 cells treated with the triterpenoid containing fraction of the present invention at different concentrations, it shows that the viability of treated cells decreases while the concentration of the triterpenoids increases.

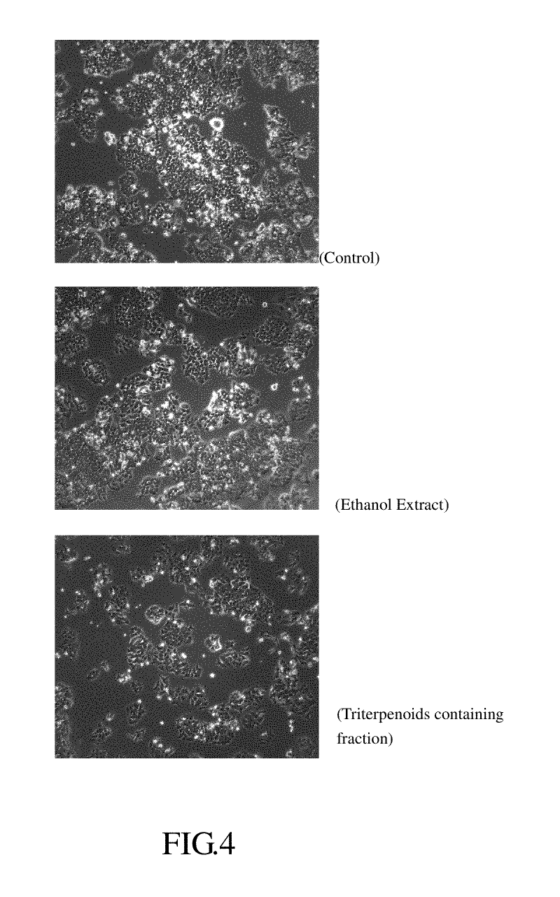

As shown in FIG. 4, it shows the changes occurred in cell morphology and viability among cells in the control group, the ethanol extract treated group, and the triterpenoid containing fraction treated group. It shows that the triterpenoid containing fraction of the present invention is significantly more effective in repressing the growth of HCT116 cells, and furthermore cells are turned rounder and deceased, which proves again that the triterpenoid containing fraction of the present invention is effective in repressing the growth of HCT116 cells.

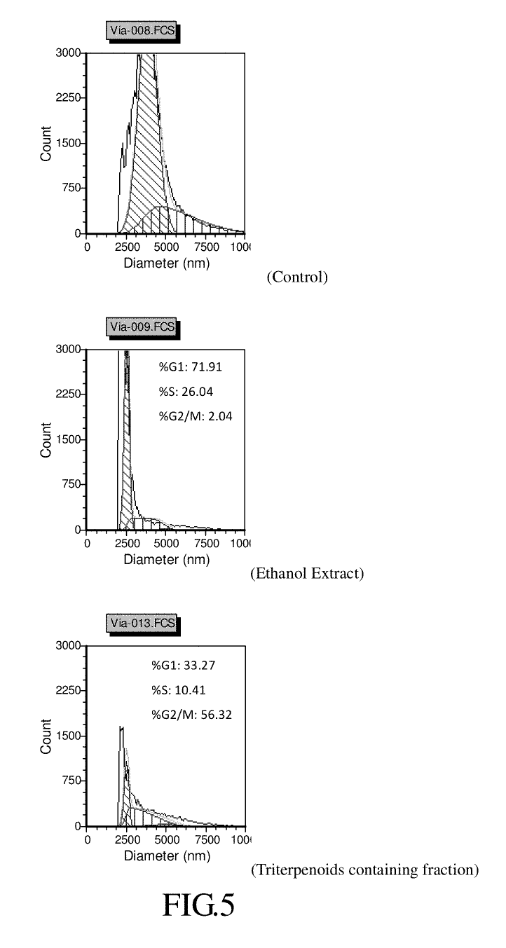

Cell cycle reflects the cell's status, cancer cells usually have abnormal division cycles that makes cancer cells grow faster than normal cells, so slowing down the cell cycle of cancer cells may be a way of anti-cancer. As shown in FIG. 5, the triterpenoid containing fraction of the present invention changes cell distribution of HCT116 cells in G2/M stage. After treatment for 24 hours, a count of HCT116 cells in G2/M stage increases, and counts of HCT116 cells at other stages decrease. In comparison with the control group, it has a significant difference in statistics (p<0.05). It is an evidence that triterpenoid containing fraction of the present invention is effective in repressing cancer cells to slow down or stop the growth of cancer cells.

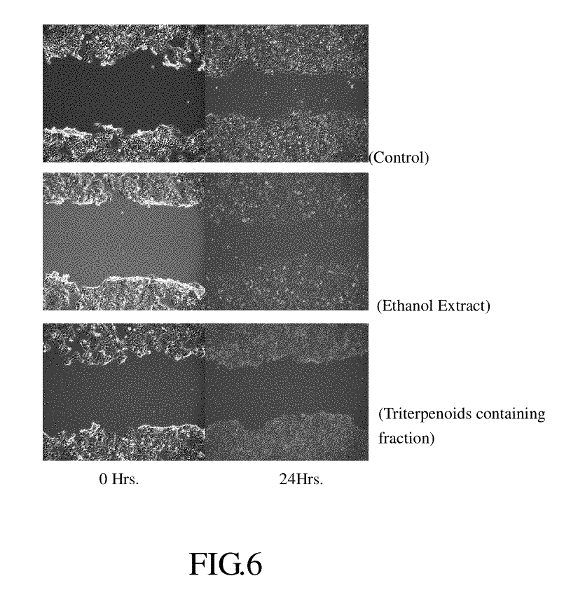

The test of wound healing is a simple and fast way to examine the cell motility, and also as a hint to tumorigenicity and metastasis of cancer cells. As shown in FIG. 6, the control group is unable to repress motility of cancer cells. In HCT116 cells treated with triterpenoid containing fraction of the present invention, the motility thereof is repressed. The result shows that triterpenoid containing fraction of the present invention is significantly more effective in repressing the motility of cancer cells than the control group.

In conclusion, the method of the preferred embodiment of the present invention provides saturated supercritical carbon dioxide as the solvent, and uses the chromatography and absorption techniques to massively separate and purify triterpenoids. It could reach the balance with less ethanol and supercritical carbon dioxide under the supercritical condition. The present invention is effective in anti-cancer that has been proved by cytotoxicity test, cell morphology analysis, cell cycle and apoptosis test, and cell motility test. The triterpenoid manufacturing method of the preferred embodiment of the present invention is a simple procedure with no residual solvent problem. It further has some advantages, including low cost, solvent recyclable, safety, and practicable.

It must be pointed out that the embodiments described above are only some preferred embodiments of the present invention. All equivalent structures which employ the concepts disclosed in this specification and the appended claims should fall within the scope of the present invention.

What is claimed is:

1. A method of making triterpenoids from petri dish cultured Antrodia cinnamomea, comprising the steps of:
A. providing 1-2 Kg petri dish cultured Antrodia cinnamomea to an extracting container;
B. providing a supercritical solvent to the extracting container to obtain an Antrodia cinnamomea extract; after 30-60 minutes, sending the Antrodia cinnamomea extract to a chromatographic column to separate impurities and triterpenoids from the Antrodia cinnamomea extract, and then removing the impurities at a bottom of the chromatographic column, and collecting the triterpenoids containing fraction at a top of the chromatographic column, wherein the supercritical solvent is a saturated mixture of supercritical carbon dioxide and ethanol with a volume ratio of 1:0.1-0.2 (v/v); a pressure of the supercritical solvent is set to 2,000-4,000 psi, a temperature is set to 40-60° C., and a flow rate is set to 3-9 L/hr; and
C. testing the bioactivity of the triterpenoids containing fraction in anti-cancer by cytotoxicity test, cell morphology analysis, cell cycle and apoptosis test, and cell motility test to evaluate anti-cancer effect of the triterpenoids.

2. The method of claim 1, wherein the cytotoxicity test includes providing HCT116 cells in a culture plate having 96 wells, in each of which 10,000 HCT116 cells and a 200 μL complete medium are received; replacing the complete medium with a 0.25-1.0 mg/mL triterpenoids medium (a medium having the triterpenoid containing fraction, and a concentration thereof is 0.25-1.0 mg/mL) for a predetermined time, and replacing the control group with only the complete medium; after a predetermined time, testing the viability of HCT116 cells in wells by a (3-(4,5-dimethylthiazol-2-yl)-2,5-diphenyltetrazolium bromide (MTT) colorimetric assay under 570 nm in optical density by an ELISA reader.

3. The method of claim 2, wherein the complete medium is replaced by the triterpenoid medium next day, and the ratio of the viable cell count is tested in 2 days later.

4. The method of claim 1, wherein the cell morphology analysis includes providing HCT116 cells in a petri dish and waiting for the HCT116 cells to attach to the bottom of the petri dish; providing a 1.0 mg/mL medium having the triterpenoids containing fraction to the petri dish and waiting for a predetermined time; and taking pictures of the HCT116 cells under an inverted microscope to observe cell morphology of the HCT116 cells.

5. The method of claim 4, wherein the HCT116 cells are attached to the bottom of the petri dish in a day later, and the triterpenoids containing fraction is received in the petri dish for 2, 7, 14, 21, and 28 days respectively.

6. The method of claim 1, wherein the cell cycle and apoptosis test includes mixing HCT116 cells with a medium having 1.0 mg/mL triterpenoids containing fraction for a predetermined time, then adding trypsin-EDTA, and collecting the medium to obtain a solution; processing the solution by centrifuging, removing a supernatant liquor thereof, washing by a phosphate buffered saline, adding 1 mL 70% cool methanol, keeping in a 4° C. environment for a predetermined time, centrifuging again, adding 1 mL phosphate buffered saline for suspension, adding 50 mg/mL propidiumiodide for a photophobic process, and exposing under 532 nm beams to test fluorescence of a 590±40 nm wavelength of the HCT116 cells by a flow cytometry.

7. The method of claim 6, wherein the photophobic process is taken for 10 minutes.

8. The method of claim 1, where in the cell motility test includes providing HCT116 cells in a culture plate having 6 wells where in each 8,000 cells are received; adding a 2 mL complete medium in the culture plate; after a predetermined time, making a line by a 200 μL pipette tip; replacing the complete medium by a medium having 0.25-1.0 mg/mL triterpenoids containing fraction; taking pictures of the HCT116 cells to measure width of the wound on Day 0 by a 100× magnification microscope; adding a 2 mL complete medium without any addition for a control group, and waiting for 24 hours; taking pictures to measure the closing of the wound on Day 1; measuring width between 5 opposite points on each pictures by a measurement software to obtain at least 25 data for each conditions to compare averages cell motility between conditions.

9. The method of claim 8, wherein the HCT116 cells is mixed with the complete medium for a day, and the measurement software is Image J.

10. Triterpenoids containing fraction, which is effective in anti-cancer, is made by the methods as defined in claim 1.

11. Triterpenoids containing fraction, which is effective in anti-cancer, is made by the methods as defined in claim 2.

12. Triterpenoids containing fraction, which is effective in anti-cancer, is made by the methods as defined in claim 4.

13. Triterpenoids containing fraction, which is effective in anti-cancer, is made by the methods as defined in claim 6.

14. Triterpenoids containing fraction, which is effective in anti-cancer, is made by the methods as defined in claim 8.
